(19) United States
(12) Patent Application Publication
(10) Pub. No.: US 2024/0207037 A1
(43) Pub. Date: Jun. 27, 2024

(54) DELIVERY SYSTEM WITH GUIDED RETRACTION MECHANISM OF A DELIVERY SYSTEM TIP

(71) Applicant: Cook Medical Technologies LLC, Bloomington, IN (US)

(72) Inventors: Kevin D. Wilger, Lafayette, IN (US); Jarin A. Kratzberg, West Lafayette, IN (US); Ryan C. Bradway, Temecula, CA (US)

(73) Assignee: Cook Medical Technologies LLC, Bloomington, IN (US)

(21) Appl. No.: 18/529,664

(22) Filed: Dec. 5, 2023

Related U.S. Application Data
(60) Provisional application No. 63/432,500, filed on Dec. 14, 2022.

(51) Int. Cl. A61F 2/07; A61F 2/95; A61M 25/09

(52) U.S. Cl. CPC A61F 2/07; A61F 2/95; A61M 25/09; A61F 2002/9511; A61M 2025/09058

(57) ABSTRACT

A delivery system for a stent graft may include a stent graft having a proximal end, a distal end, an internal lumen between the proximal end and the distal end, a graft material tube, a graft material sidewall and a plurality of longitudinally spaced apart self-expanding stents attached to the graft material sidewall; a guidewire catheter extending longitudinally through the lumen of the stent graft; and a wire extending longitudinally along only a first side of the graft material tube in an undulating pattern of successive curves in alternate directions, the wire repeatedly extending through the graft material sidewall from inside the graft material tube to outside the graft material tube such that a portion of the wire extends over the guidewire catheter along a longitudinal length of the guidewire catheter to secure the guidewire catheter at least partially to an internal wall of the stent graft.

### CROSS-REFERENCE TO RELATED APPLICATIONS

This application claims priority from U.S. Provisional Application No. 63/432,500, filed Dec. 14, 2022, which is hereby fully incorporated by reference herein in its entirety.

### TECHNICAL FIELD

This invention relates to a delivery system for intraluminal or endovascular delivery of a prosthesis.

### BACKGROUND

The deployment of a medical device, such as an endoluminal prosthesis, into the vessel of a patient from a remote location by the use of a delivery system is generally known. The term prosthesis may include, for example and without limitation, a stent, stent graft, filter, valve, balloon, embolization coil, and the like. A delivery system carrying an endoluminal prosthesis is delivered into a vessel over a guide wire previously placed within the vessel. Once the delivery system is positioned, the prosthesis is released and expanded to repair the vessel.

An endoluminal prosthesis can be used, for example, to repair diseased and/or damaged conduits, such as blood vessels, the esophagus, the trachea, and the like. Over the past decade, endoluminal prostheses have become a popular option for treating damage and disease to blood vessels, such as abdominal aortic and/or thoracic aneurysms.

In some cases, it may be necessary to deploy an endoluminal prosthesis in a major vessel (e.g., the aorta) at or near an intersecting branch vessel (e.g., innominate, carotid, subclavian, celiac, SMA, and renal arteries). In these cases, an endoluminal prosthesis may be provided with one or more fenestrations so that the prosthesis can overlap the branch vessels without blocking flow to these vessels. For example, Fenestrated and Branched Endovascular Repair (F-BEVAR) is used to treat complex aortic aneurysms such as juxtarenal, pararenal, and thoracoabdominal aneurysms. In F-BEVAR, critical vessels are perfused via bridging stents placed in the fenestration of the main body stent graft.

Once the prosthesis is placed in the main vessel, it may be necessary to provide interventional access between the main vessel and a branch vessel. For example, a physician may desire to deliver additional interventional catheters carrying balloons, stents, grafts, imaging devices, and the like through the fenestration.

Before such a catheter device can be delivered through the fenestration to a target vessel, however, a guide wire must be provided and delivered through the fenestration to the target vessel. Typically, this requires multiple steps. First, the physician must deliver and navigate a set of catheters and wires to pass a guide wire through the fenestration. Once the fenestration is cannulated, the physician must then deliver and navigate a separate set of catheters and wires to pass a guide wire into the target vessel.

Preloaded delivery systems may be used, where wires and/or catheters may be preloaded through the fenestration(s) of the delivery system. Currently, the tip of the preloaded delivery system needs to be retracted prior to the deployment of bridging stents to avoid contact with or interference by the tip with elements of the preloaded system.

### SUMMARY

One general aspect of the present disclosure includes a delivery system for a stent graft, where the delivery system includes: a stent graft having a proximal end, a distal end, a proximal portion, a distal portion, an internal lumen between the proximal end and the distal end, a graft material tube, a graft material sidewall and a plurality of longitudinally spaced apart self-expanding stents attached to the graft material sidewall and including at least an end stent and a plurality of intermediate stents; a guidewire catheter extending longitudinally through the lumen of the stent graft; and a first wire extending longitudinally along only a first side of the graft material tube in an undulating pattern of successive curves in alternate directions, the first wire repeatedly extending through the graft material sidewall from inside the graft material tube to outside the graft material tube such that a portion of the first wire extends over the guidewire catheter over and along a longitudinal length of the guidewire catheter to secure the guidewire catheter at least partially to an internal wall of the stent graft, where the first wire has a plurality of successive curves in the proximal portion and a plurality of successive curves in the distal portion, each of the plurality of successive curves in the proximal portion and the distal portion have an X-displacement and a Y-displacement, where the X-displacement of the plurality of successive curves in the distal portion is greater than the X-displacement of the plurality of successive curves in the proximal portion, and where the Y-displacement of the plurality of successive curves in the distal portion is greater than the Y-displacement of the plurality of successive curves in the proximal portion.

Another general aspect of the present disclosure includes a delivery system for a fenestrated stent graft, where the delivery system includes a stent graft having a proximal end, a distal end, a proximal portion, a distal portion, an internal lumen between the proximal end and the distal end, a graft material tube, a graft material sidewall, at least two fenestrations, and a plurality of longitudinally spaced apart self-expanding stents attached to the graft material sidewall and including at least an end stent and a plurality of intermediate stents; a guidewire catheter extending longitudinally through the lumen of the stent graft; and a first wire extending longitudinally along only a first side of the graft material tube in an undulating pattern of successive curves in alternate directions, the first wire repeatedly extending through the graft material sidewall from inside the graft material tube to outside the graft material tube such that a portion of the first wire extends over the guidewire catheter over and along a longitudinal length of the guidewire catheter to secure the guidewire catheter at least partially to an internal wall of the stent graft, where the first wire has a plurality of successive curves in the proximal portion and a plurality of successive curves in the distal portion, each of the plurality of successive curves in the proximal portion and the distal portion have an X-displacement and a Y-displacement, where the X-displacement of the plurality of successive curves in the distal portion is greater than the X-displacement of the plurality of successive curves in the proximal portion, and where the Y-displacement of the plurality of successive curves in the distal portion is greater than the Y-displacement of the plurality of successive curves in the proximal portion.

Another general aspect of the present disclosure includes a delivery system for a stent graft, where the delivery system includes a stent graft having a proximal end, a distal end, a proximal portion, a distal portion, an internal lumen between the proximal end and the distal end, a graft material tube, a graft material sidewall, at least two fenestrations, and a plurality of longitudinally spaced apart self-expanding stents attached to the graft material sidewall and including at least an end stent and a plurality of intermediate stents; a guidewire catheter extending longitudinally through the lumen of the stent graft; and a first wire extending longitudinally along only a first side of the graft material tube in an undulating pattern of successive curves in alternate directions, the first wire repeatedly extending through the graft material sidewall from inside the graft material tube to outside the graft material tube such that a portion of the first wire extends over the guidewire catheter over and along a longitudinal length of the guidewire catheter to secure the guidewire catheter at least partially to an internal wall of the stent graft, where the first wire has a plurality of successive curves in the proximal portion and a plurality of successive curves in the distal portion, each of the plurality of successive curves in the proximal portion and the distal portion have an X-displacement and a Y-displacement, where the X-displacement of the plurality of successive curves in the distal portion is greater than the X-displacement of the plurality of successive curves in the proximal portion, where the Y-displacement of the plurality of successive curves in the distal portion is greater than the Y-displacement of the plurality of successive curves in the proximal portion, and where the plurality of stents each comprise a series of struts and the first wire crosses over two adjacent struts of each of the plurality of stents at an angle to a longitudinal axis of the stent graft.

A delivery system according to the present disclosure may include any combination of the features described above and/or the original as-filed claims.

The foregoing paragraphs have been provided by way of general introduction, and are not intended to limit the scope of the following claims. The various preferred embodiments, together with further advantages, will be best understood by reference to the following detailed description taken in conjunction with the accompanying drawings.

### BRIEF DESCRIPTION OF DRAWINGS

The present disclosure can be better understood with reference to the following drawings and description. The components in the figures are not necessarily to scale, emphasis instead being placed upon illustrating the principles of the present disclosure, although certain figures may be illustrated to scale and relied upon as such. Moreover, in the figures, like-referenced numerals designate corresponding parts throughout the different views.

FIG. 1 depicts a delivery system according to one embodiment of the present invention with the sheath withdrawn to show the components underneath it.

FIG. 2 is an illustration of a stent graft with a wire extending along a length of the stent graft, according to one embodiment of the present invention, showing the stent graft including a taper disposed between the distal portion and the proximal portion of the stent graft.

FIG. 3 is an illustration of a stent graft with a wire extending along a length of the stent graft, according to one embodiment of the present invention, showing the stent graft without a taper disposed between the distal portion and the proximal portion of the stent graft.

FIG. 4 is an illustration of a stent graft with a wire extending along a length of the stent graft, according to one embodiment of the present invention, showing the stent graft including fenestrations.

FIG. 5 is an illustration of the pattern of the extension of a wire extending along a length of the stent graft, according to one embodiment of the present invention.

FIG. 6 is an illustration of the pattern of the extension of a proximal portion of a wire extending along a length of the stent graft, according to one embodiment of the present invention.

FIG. 7 is an illustration of the pattern of the extension of a distal portion of a wire extending along a length of the stent graft, according to one embodiment of the present invention.

FIG. 8 is an illustration of a stent graft coupled to a guidewire catheter of a delivery system, according to one embodiment of the present invention, showing the stent graft constrained by diameter reducing ties (DRTs) separate from the embodiments of wires shown in FIGS. 2-4.

FIG. 9 is an illustration of a stent graft with a tubular body including a scallop and fenestrations, according to one embodiment of the present invention, showing the delivery system tip held against an inner wall of the stent graft by a wire.

FIG. 10 is an illustration of a stent graft with a tubular body including fenestrations without a scallop, according to one embodiment of the present invention, showing the delivery system tip held against an inner wall of the stent graft by a wire.

FIG. 11 is an illustration of a cross-sectional view of a fenestrated stent graft, according to one embodiment of the present invention, showing the delivery system tip held away from the fenestration by a wire.

FIG. 12 is an illustration of a cross-sectional view of a delivery system for a fenestrated stent graft, according to one embodiment of the present invention, showing the delivery system tip held away from the fenestration by a wire, where the wire is coupled to a bushing in the handle of the delivery system, and the bushing is in a first position.

FIG. 13 is an illustration of a cross-sectional view of a delivery system for a fenestrated stent graft, according to one embodiment of the present invention, showing the delivery system tip held away from the fenestration by a wire, where the wire is coupled to a bushing in the handle of the delivery system, and the bushing is in a second position.

FIG. 14 is an illustration of a top view of a delivery system for a fenestrated stent graft, according to one embodiment of the present invention, showing the delivery system tip held away from the fenestrations by a wire coupled to the delivery system tip.

FIG. 15 is an illustration of a top view of a delivery system for a fenestrated stent graft, according to one embodiment of the present invention, showing the delivery system tip being removed distally.

FIG. 16 is another illustration of the pattern of the extension of a proximal portion of a wire extending along a length of the stent graft, according to one embodiment of the present invention.

FIG. 17 is another illustration of the pattern of the extension of a distal portion of a wire extending along a length of the stent graft, according to one embodiment of the present invention.

FIG. 18 is an illustration of a stent graft with a wire extending along a length of the stent graft, according to one embodiment of the present invention, showing the wire moves clockwise away from a fenestration.

FIG. 19 is an illustration of a stent graft with a wire extending along a length of the stent graft, according to one embodiment of the present invention, showing the wire moves counter clockwise away from a fenestration.

FIG. 20 is an illustration of a stent graft with a wire extending along a length of the stent graft, according to one embodiment of the present invention, showing the wire path clocks away from a fenestration.

### DETAILED DESCRIPTION

Various aspects are described below with reference to the drawings in which like elements generally are identified by like numerals. The relationship and functioning of the various elements of the aspects may better be understood by reference to the following detailed description. However, aspects are not limited to those illustrated in the drawings or explicitly described below. It also should be understood that the drawings are not necessarily to scale (although certain drawings may be drawn to scale and relied upon as such), and in certain instances details may have been omitted that are not necessary for an understanding of aspects disclosed herein, such as conventional material, construction, and assembly.

For purposes of promoting an understanding of the presently disclosed embodiments, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

Throughout this specification the term “distal” with respect to a portion of the aorta, a delivery device, or a stent graft is intended to mean the portion of the aorta, delivery device, or stent graft further away in the direction of blood flow away from the heart, and the term “proximal” is intended to mean the portion of the aorta, delivery device, or the stent graft nearer to the heart. The term “configured to” is used to describe structural limitations in a particular manner that requires specific construction to accomplish a stated function and/or to interface or interact with another component(s), and is not used to describe mere intended or theoretical uses. Relative terminology and broader terms such as “generally,” “substantially,” and the like will be understood by those of skill in the art as providing clear and definite scope of disclosure and/or claiming. For example, the term “generally vertical to the longitudinal axis 75 of the stent graft 12” will be understood as not requiring exactly 90.00 degrees to the longitudinal axis 75 of the stent graft 12, but rather including that and functional equivalents.

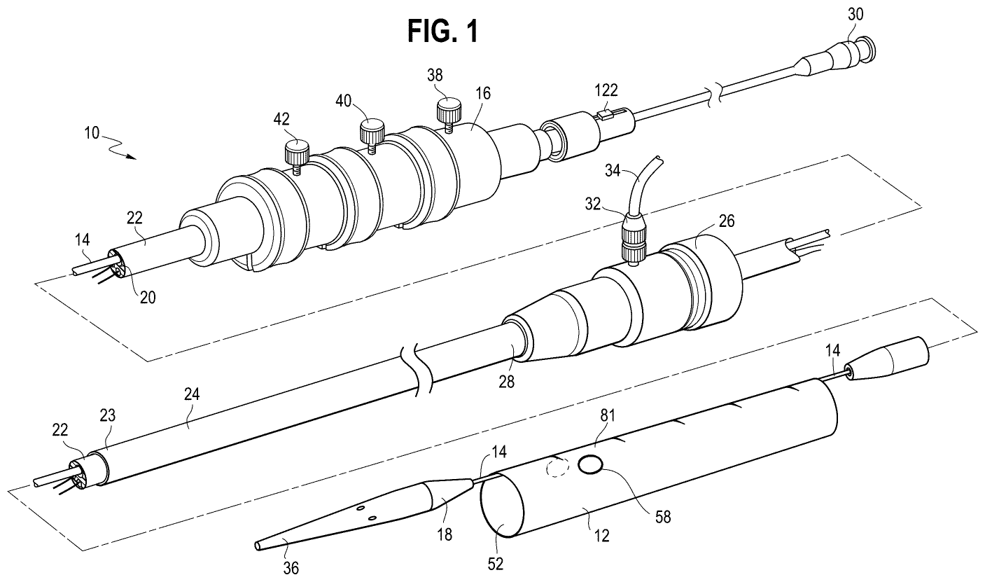

FIG. 1 depicts a delivery system 10 for a prothesis (e.g., a stent graft 12, as shown in FIG. 1) according to one embodiment of the invention. The delivery system 10 has a guidewire catheter 14 which extends from a distal handle 16 to a proximal tapered nose cone dilator 18 longitudinally through a passageway or lumen 20 of a delivery catheter 22 which is connected to the handle 16 at the distal end of the delivery catheter 22. An introducer sheath 24 fits coaxially around the delivery catheter 22 and extends from a proximal end 23 which optionally includes a radiopaque marker to a connector valve and manipulator 26 attached about the distal end 28 of the sheath 24. The introducer sheath 24 extends proximally to the nose cone dilator 18 and covers the stent graft 12 during introduction of the stent graft 12 into a patient and is withdrawn distally to expose the stent graft 12 during deployment when the stent graft 12 is in a selected position within the vasculature of a patient. The stent graft 12 is carried on the guidewire catheter 14 proximally of the delivery catheter 22 and distally of the nose cone dilator 18.

The connector valve 26 may include a silicone disk (not shown) for preventing the backflow of fluids therethrough. The disk may include a slit for the insertion of the nose cone dilator 18 and the delivery catheter 22. The connector valve 26 also includes a side arm 32 to which a tube 34 may be connected for introducing and aspirating fluids therethrough. The nose cone dilator 18 includes a tapered proximal end (e.g., delivery system tip 36) for accessing and dilating a vascular access site over a wire guide (not shown).

The wire guide is inserted in a body vessel with an introducer needle using, for example, the percutaneous vascular access Seldinger technique. A male luer lock connector hub 30 is attached at the distal end of the guidewire catheter 14 for connection to syringes and other medical apparatus. The handle 16 at the distal end of the delivery catheter 22 remains outside a patient in use and carries the trigger wire release handle mechanisms 38, 40, and 42 used to release the various portions of the stent graft 12. In some embodiments, the proximal end of the stent graft 12 may be retained on the delivery system 10 by the use of trigger wires (not shown) connected to the release handle 38. The distal end of the stent graft 12 may be retained on the delivery system 10 by the use of trigger wires (not shown) connected to the release handle 40. The longitudinal portion of the stent graft 12 may be retained on the delivery system 10 by the use of trigger wires (not shown) connected to the release handle 42.

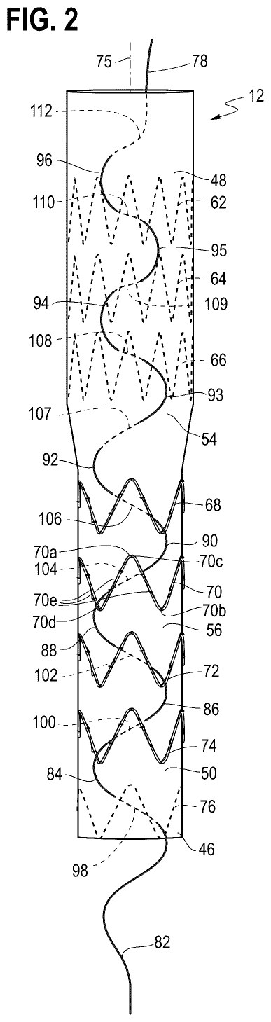

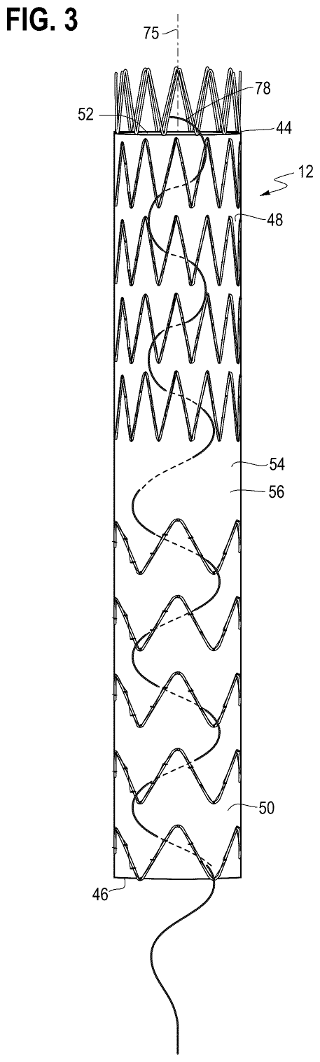

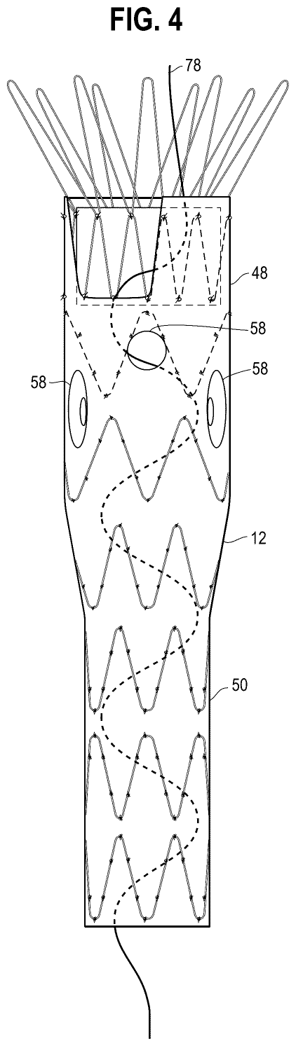

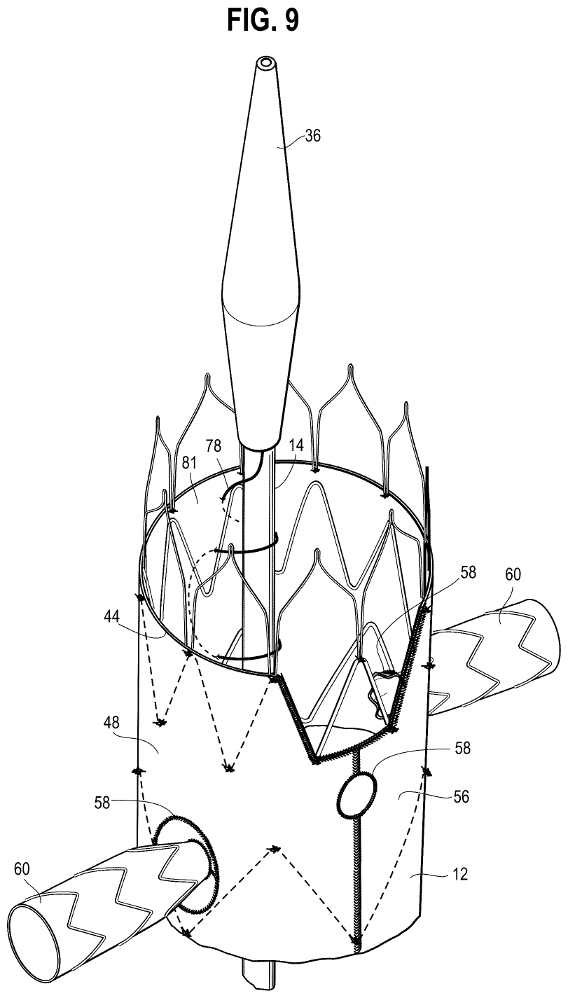

As shown in FIGS. 2 and 3, the stent graft 12 has a proximal end 44, a distal end 46, a proximal portion 48, a distal portion 50, and an internal lumen 52 between the proximal end 44 and the distal end 46. As shown in FIG. 1, the guidewire catheter 14 extends longitudinally through the lumen 52 of the stent graft 12. The stent graft 12 also includes a graft material tube 54 and a graft material sidewall 56. The sidewall 56 may have one or more openings or fenestrations 58 formed therein for providing fluid access to branch vessels, such as the renal arteries. In one example, as shown in FIG. 9, the stent graft 12 may have three fenestrations 58 located near the proximal end 44 of the stent graft 12. One or more bridging stents 60 may be deployed in one or more fenestrations 58. Different embodiments of the stent graft 12 are shown in FIGS. 2-4, and 9-10 as examples. Other embodiments of the stent graft 12 may be included in the delivery system 10, including, but not limited to, the embodiments of stent graft disclosed in U.S. Pat. Nos. 9,095,458, 9,072,621, 9,060,887, 8,523,934, 8,172,895, 8,048,140, 7,833,259, and 7,413,573, the disclosures of which are incorporated by reference in their entireties. For the sake of brevity, FIG. 2 will be used to describe various features below, and it will be understood that same/similar features described below can be incorporated into other embodiments of the stent graft 12 (e.g., the embodiments of stent graft shown in FIGS. 3 and 4).

The stent graft 12 may be any suitable selected diameter and may be constructed of any biocompatible graft material which is suitable for facilitating repair of an injured or diseased body vessel. The graft material may be synthetic and/or naturally-derived material. Synthetic biocompatible polymers may include but are not limited to polyethylene terephthalate, polyurethane, nylon, polyester, high molecular weight polyethylene, polytetrafluoroethylene, or combinations thereof. The graft material can be porous or non-porous and also may be impregnated or coated with one or more therapeutic substances. The graft material should have sufficient flexibility to allow for navigation of the vasculature and delivery to a targeted area in the body.

In some embodiments, the stent graft 12 may have a plurality of longitudinally spaced apart self-expanding stents attached to the graft material sidewall 56 and include at least an end stent and a plurality of intermediate stents. A self-expanding stent may be manufactured from a shape-memory alloy, such as nickel titanium alloy (Nitinol). If the stent comprises a self-expanding material such as Nitinol, the stent may be heat-set into the desired expanded state whereby the stent can assume a relaxed radially expanded configuration. The stent may be made from other metals and alloys that allow the stent to return to its original expanded configuration upon deployment, such as, for example, stainless steel, cobalt-chrome alloys, amorphous metals, and/or non-metallic materials as would be recognized by one of ordinary skill in the art.

One or more stents may be located on an interior surface, exterior surface, or both of the graft material tube 54 of the stent graft 12. In some embodiments, the proximal portion 48 of the stent graft 12 may have at least two adjacent stents (e.g., stents 62 and 64, as shown in FIG. 2) and the distal portion 50 of the stent graft 12 may have at least two adjacent stents (e.g., stents 70 and 72, as shown in FIG. 2), where a distance between the at least two adjacent stents (e.g., stents 62 and 64, as shown in FIG. 2) of the proximal portion 48 may be less than, equal to, or greater than the distance between the at least two adjacent stents (e.g., stents 70 and 72, as shown in FIG. 2) of the distal portion 50.

For example, as shown in FIG. 2, the stent graft 12 has four internal stents 62, 64, 66, and 76 shown in dotted lines and four external stents 68, 70, 72, and 74 along the length of its tubular body, the stents 64, 66, 68, 70, 72, and 74 being intermediate stents and the stents 62 and 76 being end stents. The internal stents 62, 64, and 66 are in the proximal portion 48 of the stent graft 12 and the external stents 68, 70, 72, 74 and the internal stent 76 are in the distal portion 50 of the stent graft 12. The number of internal stents and external stents in the proximal and distal portions 48 and 50 and the distance between adjacent stents may be varied, as desired and/or needed, without departing from the scope of the present invention.

In some embodiments, the stents may be in a ring configuration. In one example, as shown in FIG. 2, each stent may be a Z-stent, where each stent (e.g., stent 70) may have a proximal end (e.g., 70a) with a series of proximal apices (e.g., 70c) and a distal end (e.g., 70b) with a series of distal apices (e.g., 70d). Each stent (e.g., stent 70) may also have one or more elongate struts (e.g., 70e) connecting the proximal apices (e.g., 70c) to the distal apices (e.g., 70d).

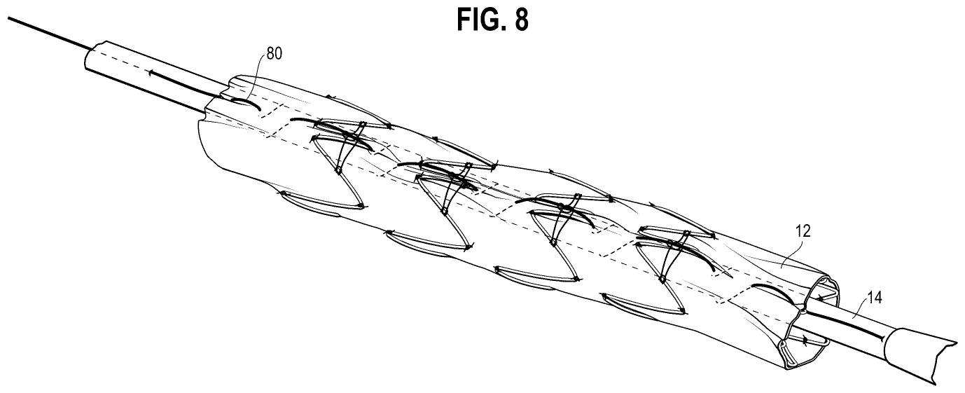

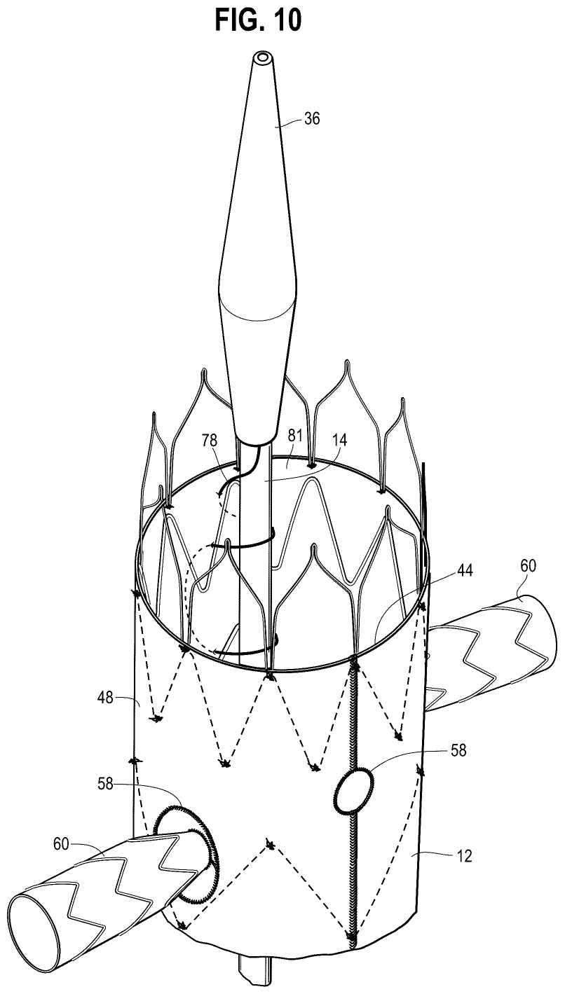

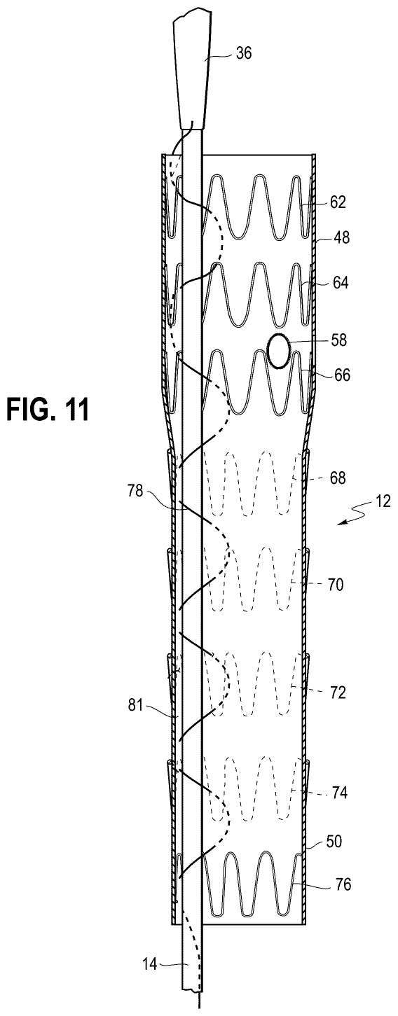

In some embodiments, as shown in FIGS. 2-4, the delivery system 10 may also include a wire 78 (separate from the diameter reducing ties 80 (DRTs) of the delivery system 10, e.g., as shown in FIG. 8) extending longitudinally along only a first side of the graft material tube 54 in an undulating pattern of successive curves in alternate directions. An embodiment of the diameter reducing ties (DRTs) is shown in FIG. 8 as an example. Other embodiments of the diameter reducing ties (DRTs) may be included in the delivery system 10, including, but not limited to, the embodiments of diameter reducing ties (DRTs) disclosed in U.S. Pat. Nos. 9,504,555, 9,707,072, and 8,728,148, the disclosures of which are incorporated by reference in their entireties. The wire 78 may be made of a flexible material, such as nickel titanium alloy (Nitinol, e.g., 0.007-0.010 Nitinol). The wire 78 repeatedly extends through the graft material sidewall 56 from inside the graft material tube 54 to outside the graft material tube 54 such that a portion of the wire 78 extends over the guidewire catheter 14 over and along a longitudinal length of the guidewire catheter 14 to secure the guidewire catheter 14 at least partially to an internal wall 81 of the stent graft 12 (e.g., as shown in FIG. 11).

For example, referring to FIGS. 2 and 11, in the distal portion 50 of the stent graft, the wire 78 passes through the graft material sidewall 56 into the lumen 52 of the stent graft 12, traverses across (e.g., from right to left) two struts of the stent 76 and the guidewire catheter 14 at a first angle (e.g., acute angle) to a longitudinal axis 75 of the stent graft 12. The wire 78 then passes through the graft material sidewall 56 to outside the graft material tube 54, extends proximally, and then passes through the graft material sidewall 56 into the lumen 52 of the stent graft 12, traverses across (e.g., from left to right) two struts of the stent 74 and the guidewire catheter 14 at a second angle (e.g., acute angle) to the longitudinal axis of the stent graft 12. The wire 78 then passes through the graft material sidewall 56 to outside the graft material tube 54, extends proximally, and then passes through the graft material sidewall 56 into the lumen 52 of the stent graft 12, traverses across (e.g., from right to left) two struts of the stent 72 and the guidewire catheter 14 at a third angle (e.g., acute angle) to the longitudinal axis 75 of the stent graft 12.

The wire 78 then passes through the graft material sidewall 56 to outside the graft material tube 54, extends proximally, and then passes through the graft material sidewall 56 into the lumen 52 of the stent graft 12, traverses across (e.g., from left to right) two struts of the stent 70 and the guidewire catheter 14 at a fourth angle (e.g., acute angle) to the longitudinal axis 75 of the stent graft 12. The wire 78 then passes through the graft material sidewall 56 to outside the graft material tube 54, extends proximally, and then passes through the graft material sidewall 56 into the lumen 52 of the stent graft 12, traverses across (e.g., from right to left) two struts of the stent 68 and the guidewire catheter 14 at a fifth angle (e.g., acute angle) to the longitudinal axis 75 of the stent graft 12. The first, second, third, fourth, and fifth angles may be the same or different, and the cross-over sections of the wire 78 at each stent (68-76) may be longitudinally aligned or offset, without departing from the scope of the present invention.

Then, the wire 78 continues to extend proximally to the proximal portion 48 of the stent graft 12. The wire 78 passes through the graft material sidewall 56 into the lumen 52 of the stent graft 12, extends proximally, and then passes through the graft material sidewall 56 to outside the graft material tube 54. The wire 78 then extends proximally, passes through the graft material sidewall 56 into the lumen 52 of the stent graft 12, traverses across (e.g., from right to left) two struts of the stent 66 and the guidewire catheter 14 at a sixth angle (e.g., acute angle) to the longitudinal axis of the stent graft 12. The wire 78 then passes through the graft material sidewall 56 to outside the graft material tube 54, extends proximally, and then passes through the graft material sidewall 56 into the lumen 52 of the stent graft 12, traverses across (e.g., from left to right) two struts of the stent 64 and the guidewire catheter 14 at a seventh angle (e.g., acute angle) to the longitudinal axis 75 of the stent graft 12.

The wire 78 then passes through the graft material sidewall 56 to outside the graft material tube 54, extends proximally, and then passes through the graft material sidewall 56 into the lumen 52 of the stent graft 12, traverses across (e.g., from right to left) two struts of the stent 62 and the guidewire catheter 14 at an eighth angle (e.g., acute angle) to the longitudinal axis 75 of the stent graft 12. The sixth, seventh, and eighth angles may be the same or different, and the cross-over sections of the wire 78 at each stent (62-66) may be longitudinally aligned or offset, without departing from the scope of the present invention. It will be appreciated that the wire diameter, wire material, pitch of the wire (e.g., run from right to left or run from left to right), number of the cross-over sections, number of the internal wire portions, and location of the internal wire portions may be varied, as desired and/or needed, without departing from the scope of the present invention, as long as the guidewire catheter 14 is held by the wire 78 against a selected longitudinal portion of the stent graft 12 in the lumen 52 of the stent graft 12.

The wire 78 repeatedly penetrates the graft material sidewall 56 from inside the graft material tube 54 to outside the graft material tube 54 and then penetrate the graft material sidewall 56 back from outside the graft material tube 54 to inside the graft material tube 54, thereby forming a plurality of external wire portions and internal wire portions (e.g., cross-over sections) as a result of the repeated penetrations. For example, as shown in FIG. 2, external wire portions 82, 84, 86, 88, 90, 92, 93, 94, 95, and 96, and internal wire portions 98, 100, 102, 104, 106, 107, 108, 109, 110, and 112 may be created. In some embodiments, as shown in FIG. 2, each stent has an internal wire portion. It will be appreciated that the number of internal wire portions that each stent has may be varied, as desired and/or needed, without departing from the scope of the present invention. For example, some stents may have 2, 3, or 4, etc. internal wire portions, some stents may have no internal wire portions, and different stents may have the same number or different numbers of internal wire portions.

In some embodiments, referring to FIGS. 2 and 11, the wire 78 may repeatedly cross over the guidewire catheter 14 in the distal portion 50 of the stent graft 12, forming a distal path in the lumen 52 of the stent graft 12 by the internal wire portions in the distal portion 50 of the stent graft 12 (e.g., internal wire portions 98, 100, 102, 104, and 106, as shown in FIG. 2). The wire 78 may repeatedly cross over the guidewire catheter 14 in the proximal portion 48 of the stent graft 12, forming a proximal path in the lumen 52 of the stent graft 12 by the internal wire portions in the proximal portion 48 of the stent graft 12 (e.g., internal wire portions 108, 109, 110, and 112, as shown in FIG. 2). In some embodiments, the distal path may be longitudinally offset from the proximal path. In some embodiments, as shown in FIG. 2, the wire 78 may be disposed substantially externally along the stent graft 12. The distal path and the proximal path formed by the wire 78 in the lumen 52 of the stent graft 12 holds the guidewire catheter 14 against a selected longitudinal portion of the stent graft 12, as described in greater detail below.

In some embodiments, the plurality of stents each may have a series of struts and the wire 78 may cross over two adjacent struts (e.g., struts 70e of the stent 70) and one apex (e.g., the apex 70c of the stent 70) of each of the plurality of stents at an angle (e.g., an acute angle) to the longitudinal axis 75 of the stent graft 12. For example, as shown in FIG. 2, the wire 78 crosses over the two adjacent struts 70e within the lumen 52 of the stent graft 12. It will be appreciated that the number of struts of each stent that the wire 78 crosses over may be varied (e.g., the wire 78 crosses over a single stent strut of a stent, crosses over no stent strut of a stent, or crosses over different numbers of stent struts of different stents), as desired and/or needed, without departing from the scope of the present invention.

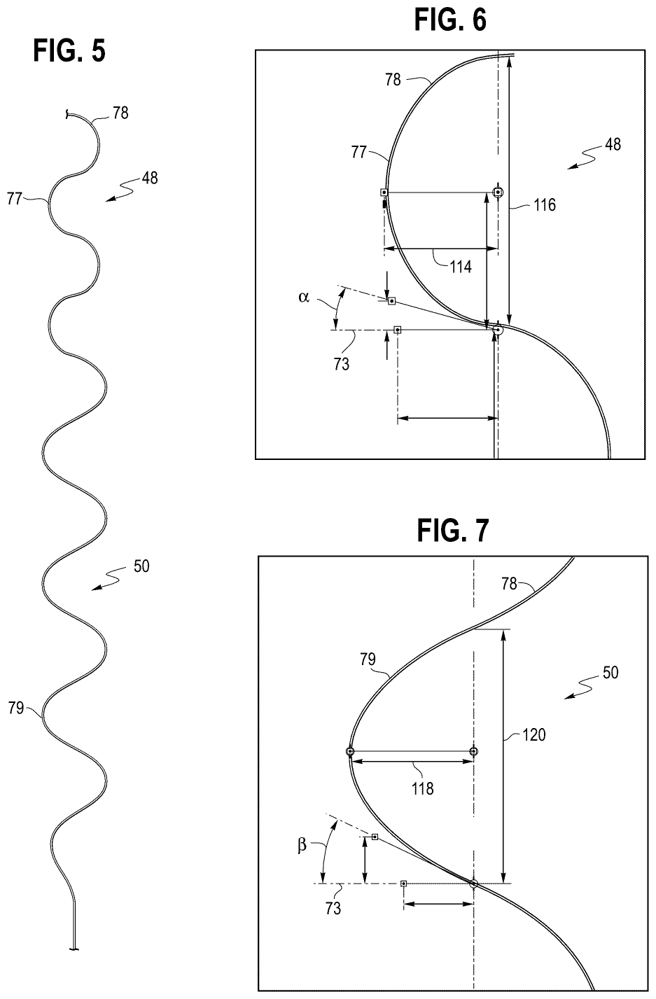

Referring to FIGS. 5-7, the wire 78 has a plurality of successive curves 77 in the proximal portion 48 of the stent graft 12 and a plurality of successive curves 79 in the distal portion 50 of the stent graft 12. Each of the plurality of successive curves 77 in the proximal portion 48 has an X-displacement 114 and a Y-displacement 116, and each of the plurality of successive curves 79 in the distal portion 50 has an X-displacement 118 and a Y-displacement 120. In some embodiments, as shown in FIGS. 6 and 7, the X-displacement 118 of the plurality of successive curves 79 in the distal portion 50 is greater than the X-displacement 114 of the plurality of successive curves 77 in the proximal portion 48, and the Y-displacement 120 of the plurality of successive curves 79 in the distal portion 50 is greater than the Y-displacement 116 of the plurality of successive curves 77 in the proximal portion 48. For example, The X-displacement 114 of each of the plurality of successive curves 77 in the proximal portion 48 may be 8±2 mm, the X-displacement 118 of each of the plurality of successive curves 79 in the distal portion 50 may be 10.5±2 mm, the Y-displacement 116 of each of the plurality of successive curves 77 in the proximal portion 48 may be 20±2 mm, and the Y-displacement 120 of each of the plurality of successive curves 79 in the distal portion 50 may be 21.5±2 mm.

It will be appreciated that in some embodiments, the X-displacement 118 of the plurality of successive curves 79 in the distal portion 50 may be smaller than the X-displacement 114 of the plurality of successive curves 77 in the proximal portion 48, and the Y-displacement 120 of the plurality of successive curves 79 in the distal portion 50 may be smaller than the Y-displacement 116 of the plurality of successive curves 77 in the proximal portion 48, without departing from the scope of the present invention. Having the Y-displacement 116 of the plurality of successive curves 77 in the proximal portion 48 greater than the X-displacement 114 of the plurality of successive curves 77 in the proximal portion 48 is advantageous for making sure the curves 77 are not too stiff. Similarly, having the Y-displacement 120 of the plurality of successive curves 79 in the distal portion 50 greater than the X-displacement 118 of the plurality of successive curves 79 in the distal portion 50 is advantageous for making sure the curves 79 are not too stiff.

In some embodiments, as shown in FIGS. 6 and 7, each of the plurality of successive curves 77 in the proximal portion 48 has a take off angle α (e.g., the angle at which the wire 78 crosses over the struts of a stent in the proximal portion 48 relative to a direction 73 generally vertical (e.g., 85-95 degrees) to the longitudinal axis 75 of the stent graft 12), and each of the plurality of successive curves 79 in the distal portion 50 has a take off angle β (e.g., the angle at which the wire 78 crosses over the struts of a stent in the distal portion 50 relative to a direction 73 generally vertical (e.g., 85-95 degrees) to the longitudinal axis 75 of the stent graft 12). In some embodiments, as shown in FIGS. 6 and 7, the take off angle β of at least one of the plurality of successive curves 79 in the distal portion 50 is greater than the take off angle α of at least one of the plurality of successive curves 77 in the proximal portion 48. For example, the take off angle β of at least one of the plurality of successive curves 79 in the distal portion 50 may be 25±5 degrees, and the take off angle α of at least one of the plurality of successive curves 77 in the proximal portion 48 may be 15±5 degrees. In some embodiments, the take off angle β of at least one of the plurality of successive curves 79 in the distal portion 50 may be smaller than the take off angle α of at least one of the plurality of successive curves 77 in the proximal portion 48.

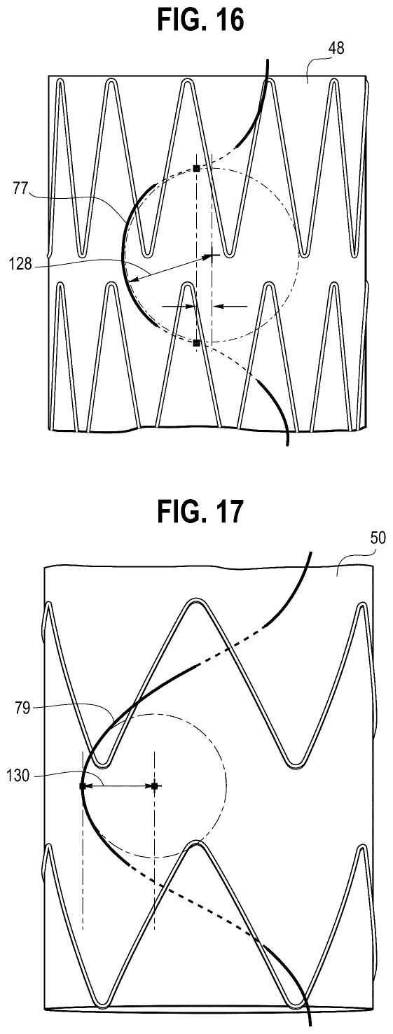

In some embodiments, as shown in FIGS. 16 and 17, each of the plurality of successive curves 77 in the proximal portion 48 may have a radius of curvature 128 and each of the plurality of successive curves 79 in the distal portion 50 may have a radius of curvature 130, and the radius of curvature 128 of at least one of the plurality of successive curves 77 in the proximal portion 48 may be greater or smaller than the radius of curvature 130 of at least one of the plurality of successive curves 79 in the distal portion 50. In some embodiments, the radius of curvature 128 of at least one of the plurality of successive curves 77 in the proximal portion 48 may range between 4-10 mm, preferably between 5-8 mm, and more preferably between 6-7 mm. In some embodiments, the radius of curvature 130 of at least one of the plurality of successive curves 79 in the distal portion 50 may range between 5-15 mm, preferably between 7-12 mm, and more preferably between 9-10 mm.

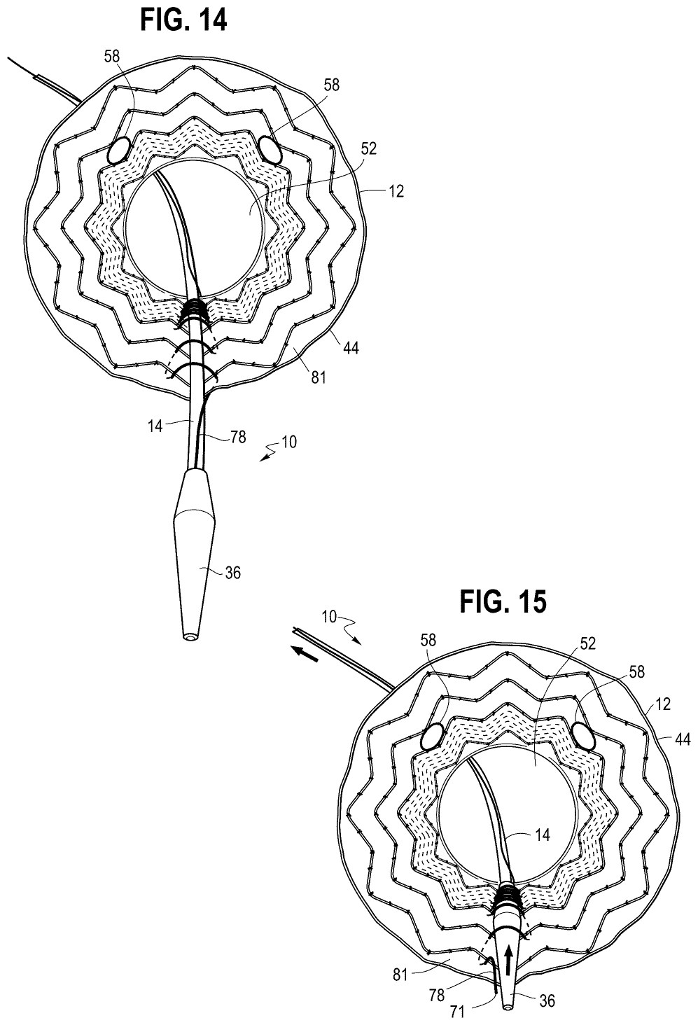

Referring to FIGS. 4 and 9-11, in some embodiments, the stent graft 12 may be a fenestrated stent graft (e.g., including at least two fenestrations), and the wire 78 may repeatedly cross over the guidewire catheter 14 along a length of the guidewire catheter 14 to secure the guidewire catheter 14 at least partially to the internal wall 81 of the stent graft 12 and to keep the guidewire catheter 14 away from the fenestrations 58 (e.g., three fenestrations 58, as shown in FIGS. 4, 9, and 10) of the stent graft 12. For example, as shown in FIGS. 4 and 9-11, the proximal end 71 (e.g., as shown in FIG. 15) of the wire 78 is releasably attached to the proximal end (e.g., delivery system tip 36) of the nose cone dilator 18. The internal wall 81 is circumferentially offset from the fenestrations 58. The wire 78 repeatedly crosses over the guidewire catheter 14 and constrains the guidewire catheter 14 further from the fenestrations 58.

This arrangement of the wire 78 is advantageous for constraining and controlling the guidewire catheter 14 traveling through the inner lumen 52 of the stent graft 12 from unwanted movement. For example, when one or more bridging stents 60 are deployed in one or more fenestrations 58, the guidewire catheter 14 is constrained by the internal wire portions formed by the wire 78 such that the guidewire catheter 14 is less likely to have unwanted incidental contact with the bridging stent(s) 60 that may be disposed in the lumen 52 of the stent graft 12, and therefore less likely to cause undesired movement or damage (e.g. denting or compression) to the bridging stent(s) 60. Currently, to avoid the unwanted incidental contact with the bridging stents 60 by the guidewire catheter 14, the delivery system 10 (with the delivery system tip 36) is retracted prior to the deployment of the bridging stent(s) 60 to avoid contact/damage from the tip 36. With the arrangement of the wire 78 disclosed in this application, the delivery and deployment process can be simplified by removing the additional step to retract the tip 36 prior to deployment of bridging stent(s), while protecting bridging stent integrity and patency. It will be appreciated that the arrangement of the wire 78 disclosed above may be incorporated in other types of stent grafts, which may or may not have fenestrations, to constrain and control the movement of the guidewire catheter 14, as discussed above.

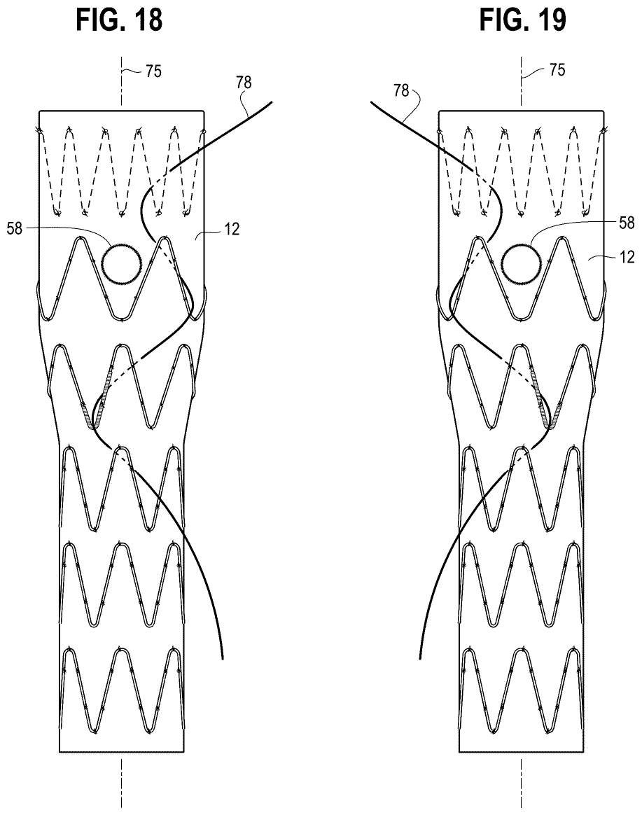

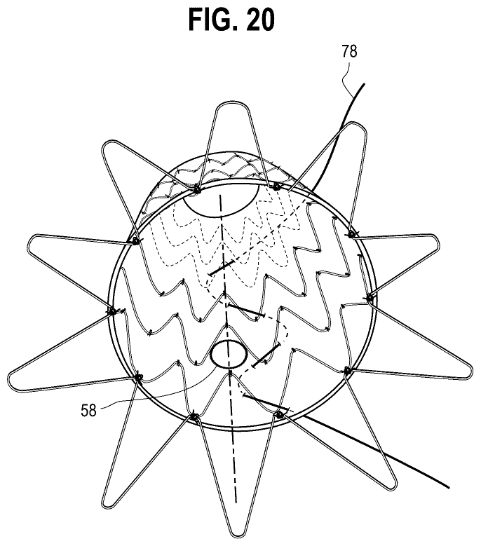

In some embodiments, as shown in FIGS. 18-20, the wire 78 may move clockwise (e.g., FIG. 18) or counter clockwise (e.g., FIG. 19) when moving up a stent or down a stent, such that the wire path clocks to the left or right around a fenestration 58 (e.g., the wire 78 does not traverses across the fenestration 58). As shown, in these embodiments, the stents that the wire 78 traverses across are not disposed along a line that is parallel to the longitudinal axis 75 of the stent graft 12.

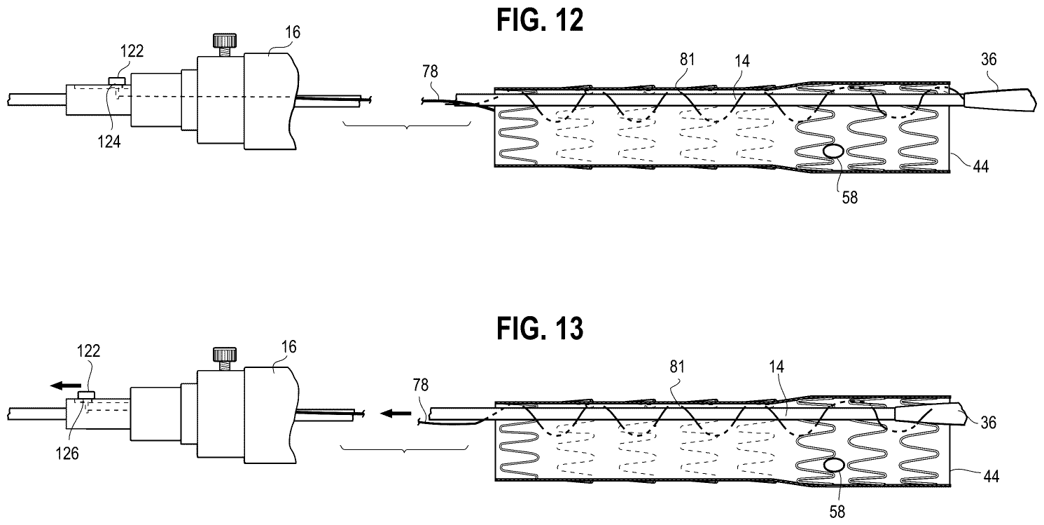

Referring to FIGS. 12-15, the wire 78 runs from above the proximal end 44 of the stent graft 12, into the trigger wire holes in the multi-lumen positioner (not shown), and out of the delivery system handle 16 and is constrained by (e.g., attached to) a bushing 122. In some embodiments, the bushing 122 may be made of stainless steel or other types of metals, and may be in the form of a threaded female standoff. The bushing 122 is configured to be movable along a distance of the handle 16 (e.g., movable between a first position 124 and a second position 126, as shown in FIGS. 12 and 13), which allows for some slack between the travel of the tip 36 and the wire 78.

Referring to FIGS. 14 and 15, as the entire delivery system 10 is removed (e.g., retrieved distally), the tip 36 is retracted through the path formed by the wire 78, and the wire 78 is also retracted from the stent graft 12. The internal wire portions (e.g., 98, 100, 102, 104, 106, 107, 108, 109, 110, and 112, as shown in FIG. 11) in the path formed by the wire 78 in the lumen 52 of the stent graft 12 are configured to increase in diameter (e.g., getting loose) and allow for the tip 36 to pass, but still constrain the tip 36 to the internal wall 81 of the stent graft 12. The ability of the internal wire portions formed by the wire 78 to increase in diameter while still constraining the tip 36 is advantageous for keeping the tip 36 and/or the guidewire catheter 14 away from the fenestrations 58 and other component(s) disposed in the lumen 52 of the stent graft 12, which may extend through the fenestrations 58, thereby reducing undesired contact or damage to the components by the tip 36 and/or the guidewire catheter 14. It will be appreciated that the wire diameter, wire material, pitch of the wire, number of the internal wire portions, location of the internal wire portions, and the slack between the travel of the tip 36 and the wire 78 may be varied as desired and/or needed, without departing from the scope of the present invention, as long as the internal wire portions formed by the wire 78 increase in diameter while still constraining the tip 36 when the delivery system 10 is removed.

While various embodiments of the present disclosure have been described, the present disclosure is not to be restricted except in light of the attached claims and their equivalents. One skilled in the relevant art will recognize that numerous variations and modifications may be made to the embodiments described above without departing from the scope of the present invention, as defined by the appended claims. Moreover, the advantages described herein are not necessarily the only advantages of the present disclosure and it is not necessarily expected that every embodiment of the present disclosure will achieve all of the advantages described.

We claim:

1. A delivery system for a stent graft, the delivery system comprising:
a stent graft having a proximal end, a distal end, a proximal portion, a distal portion, an internal lumen between the proximal end and the distal end, a graft material tube, a graft material sidewall and a plurality of longitudinally spaced apart self-expanding stents attached to the graft material sidewall and including at least an end stent and a plurality of intermediate stents;
a guidewire catheter extending longitudinally through the lumen of the stent graft; and
a first wire extending longitudinally along only a first side of the graft material tube in an undulating pattern of successive curves in alternate directions, the first wire repeatedly extending through the graft material sidewall from inside the graft material tube to outside the graft material tube such that a portion of the first wire extends over the guidewire catheter over and along a longitudinal length of the guidewire catheter to secure the guidewire catheter at least partially to an internal wall of the stent graft,
wherein the first wire has a plurality of successive curves in the proximal portion and a plurality of successive curves in the distal portion, each of the plurality of successive curves in the proximal portion and the distal portion have an X-displacement and a Y-displacement, wherein the X-displacement of the plurality of successive curves in the distal portion is greater than the X-displacement of the plurality of successive curves in the proximal portion, and wherein the Y-displacement of the plurality of successive curves in the distal portion is greater than the Y-displacement of the plurality of successive curves in the proximal portion.

2. The delivery system of claim 1, wherein each of the plurality of successive curves in the proximal portion and the distal portion has a take off angle, wherein the take off angle of at least one of the plurality of successive curves in the distal portion is greater than the take off angle of at least one of the plurality of successive curves in the proximal portion.

3. The delivery system of claim 1, wherein each of the plurality of successive curves in the proximal portion has a radius of curvature and each of the plurality of successive curves in the distal portion has a radius of curvature, wherein the radius of curvature of at least one of the plurality of successive curves in the proximal portion is less than the radius of curvature of at least one of the plurality of successive curves in the distal portion.

4. The delivery system of claim 1, wherein the proximal portion has at least two adjacent stents and the distal portion has at least two adjacent stents, wherein a distance between the at least two adjacent stents of the proximal portion is less than the distance between the at least two adjacent stents of the distal portion.

5. The delivery system of claim 1, wherein the plurality of stents each comprise a series of struts and the first wire crosses over two adjacent struts of each of the plurality of stents at an angle to a longitudinal axis of the stent graft.

6. The delivery system of claim 5, wherein the first wire crosses over the two adjacent struts within the lumen of the stent graft.

7. The delivery system of clam 1, wherein the first wire is disposed substantially externally along the stent graft.

8. The delivery system of claim 1, further comprising a second wire extending longitudinally along the first side of the graft material tube in an undulating pattern of successive curves in alternate directions, which undulating pattern of successive curves of the second wire is opposite the undulating pattern of successive curves of the first wire and repeatedly overlaps the first wire, the second wire repeatedly extending through the graft material sidewall from inside the graft material tube to outside the graft material tube such that a portion of the second wire extends over the guidewire catheter over and along a longitudinal length of the guidewire catheter to secure the guidewire catheter at least partially to an internal wall of the stent graft,
wherein the second wire has a plurality of successive curves in the proximal portion and a plurality of successive curves in the distal portion, each of the plurality of successive curves in the proximal portion and the distal portion have an X-displacement and a Y-displacement, wherein the X-displacement of the plurality of successive curves in the distal portion is greater than the X-displacement of the plurality of successive curves in the proximal portion, and wherein the Y-displacement of the plurality of successive curves in the distal portion is greater than the Y-displacement of the plurality of successive curves in the proximal portion.

9. A delivery system for a fenestrated stent graft, the delivery system comprising:
a stent graft having a proximal end, a distal end, a proximal portion, a distal portion, an internal lumen between the proximal end and the distal end, a graft material tube, a graft material sidewall, at least two fenestrations, and a plurality of longitudinally spaced apart self-expanding stents attached to the graft material sidewall and including at least an end stent and a plurality of intermediate stents;
a guidewire catheter extending longitudinally through the lumen of the stent graft; and
a first wire extending longitudinally along only a first side of the graft material tube in an undulating pattern of successive curves in alternate directions, the first wire repeatedly extending through the graft material sidewall from inside the graft material tube to outside the graft material tube such that a portion of the first wire extends over the guidewire catheter over and along a longitudinal length of the guidewire catheter to secure the guidewire catheter at least partially to an internal wall of the stent graft,
wherein the first wire has a plurality of successive curves in the proximal portion and a plurality of successive curves in the distal portion, each of the plurality of successive curves in the proximal portion and the distal portion have an X-displacement and a Y-displacement, wherein the X-displacement of the plurality of successive curves in the distal portion is greater than the X-displacement of the plurality of successive curves in the proximal portion, and wherein the Y-displacement of the plurality of successive curves in the distal portion is greater than the Y-displacement of the plurality of successive curves in the proximal portion.

10. The delivery system of claim 9, wherein each of the plurality of successive curves in the proximal portion and the distal portion has a take off angle, wherein the take off angle of at least one of the plurality of successive curves in the distal portion is greater than the take off angle of at least one of the plurality of successive curves in the proximal portion.

11. The delivery system of claim 9, wherein each of the plurality of successive curves in the proximal portion has a radius of curvature and each of the plurality of successive curves in the distal portion has a radius of curvature, wherein the radius of curvature of at least one of the plurality of successive curves in the proximal portion is less than the radius of curvature of at least one of the plurality of successive curves in the distal portion.

12. The delivery system of claim 9, wherein the plurality of stents each comprise a series of struts and the first wire crosses over two adjacent struts of each of the plurality of stents at an angle to a longitudinal axis of the stent graft.

13. The delivery system of claim 12, wherein the first wire crosses over the two adjacent struts within the lumen of the stent graft.

14. The delivery system of clam 9, wherein the first wire is disposed substantially externally along the stent graft.

15. The delivery system of clam 9, wherein the first wire repeatedly crosses over the guidewire catheter along a length of the guidewire catheter to secure the guidewire catheter at least partially to the internal wall of the stent graft and to keep the guidewire catheter away from fenestrations of the stent graft.

16. The delivery system of claim 9, further comprising a second wire extending longitudinally along the first side of the graft material tube in an undulating pattern of successive curves in alternate directions, which undulating pattern of successive curves of the second wire is opposite the undulating pattern of successive curves of the first wire and repeatedly overlaps the first wire, the second wire repeatedly extending through the graft material sidewall from inside the graft material tube to outside the graft material tube such that a portion of the second wire extends over the guidewire catheter over and along a longitudinal length of the guidewire catheter to secure the guidewire catheter at least partially to an internal wall of the stent graft,
wherein the second wire has a plurality of successive curves in the proximal portion and a plurality of successive curves in the distal portion, each of the plurality of successive curves in the proximal portion and the distal portion have an X-displacement and a Y-displacement, wherein the X-displacement of the plurality of successive curves in the distal portion is greater than the X-displacement of the plurality of successive curves in the proximal portion, and wherein the Y-displacement of the plurality of successive curves in the distal portion is greater than the Y-displacement of the plurality of successive curves in the proximal portion.

17. A delivery system for a stent graft, the delivery system comprising:
a stent graft having a proximal end, a distal end, a proximal portion, a distal portion, an internal lumen between the proximal end and the distal end, a graft material tube, a graft material sidewall, at least two fenestrations, and a plurality of longitudinally spaced apart self-expanding stents attached to the graft material sidewall and including at least an end stent and a plurality of intermediate stents;
a guidewire catheter extending longitudinally through the lumen of the stent graft; and
a first wire extending longitudinally along only a first side of the graft material tube in an undulating pattern of successive curves in alternate directions, the first wire repeatedly extending through the graft material sidewall from inside the graft material tube to outside the graft material tube such that a portion of the first wire extends over the guidewire catheter over and along a longitudinal length of the guidewire catheter to secure the guidewire catheter at least partially to an internal wall of the stent graft,
wherein the first wire has a plurality of successive curves in the proximal portion and a plurality of successive curves in the distal portion, each of the plurality of successive curves in the proximal portion and the distal portion have an X-displacement and a Y-displacement, wherein the X-displacement of the plurality of successive curves in the distal portion is greater than the X-displacement of the plurality of successive curves in the proximal portion, wherein the Y-displacement of the plurality of successive curves in the distal portion is greater than the Y-displacement of the plurality of successive curves in the proximal portion, and
wherein the plurality of stents each comprise a series of struts and the first wire crosses over two adjacent struts of each of the plurality of stents at an angle to a longitudinal axis of the stent graft.

18. The delivery system of claim 17, wherein the first wire crosses over the two adjacent struts within the lumen of the stent graft.

19. The delivery system of claim 17, wherein each of the plurality of successive curves in the proximal portion and the distal portion has a take off angle, wherein the take off angle of at least one of the plurality of successive curves in the distal portion is greater than the take off angle of at least one of the plurality of successive curves in the proximal portion.

20. The delivery system of claim 17, wherein each of the plurality of successive curves in the proximal portion has a radius of curvature and each of the plurality of successive curves in the distal portion has a radius of curvature, wherein the radius of curvature of at least one of the plurality of successive curves in the proximal portion is less than the radius of curvature of at least one of the plurality of successive curves in the distal portion.

21. The delivery system of clam 17, wherein the first wire repeatedly crosses over the guidewire catheter along a length of the guidewire catheter to secure the guidewire catheter at least partially to the internal wall of the stent graft and to keep the guidewire catheter away from fenestrations of the stent graft.

22. The delivery system of clam 17, wherein the first wire repeatedly crosses over the guidewire catheter in the distal portion of the stent graft, forming a distal path in the lumen of the stent graft, wherein the first wire repeatedly crosses over the guidewire catheter in the proximal portion of the stent graft, forming a proximal path in the lumen of the stent graft, and wherein the distal path is longitudinally offset from the proximal path.

23. The delivery system of claim 17, further comprising a second wire extending longitudinally along the first side of the graft material tube in an undulating pattern of successive curves in alternate directions, which undulating pattern of successive curves of the second wire is opposite the undulating pattern of successive curves of the first wire and repeatedly overlaps the first wire, the second wire repeatedly extending through the graft material sidewall from inside the graft material tube to outside the graft material tube such that a portion of the second wire extends over the guidewire catheter over and along a longitudinal length of the guidewire catheter to secure the guidewire catheter at least partially to an internal wall of the stent graft,
wherein the second wire has a plurality of successive curves in the proximal portion and a plurality of successive curves in the distal portion, each of the plurality of successive curves in the proximal portion and the distal portion have an X-displacement and a Y-displacement, wherein the X-displacement of the plurality of successive curves in the distal portion is greater than the X-displacement of the plurality of successive curves in the proximal portion, and wherein the Y-displacement of the plurality of successive curves in the distal portion is greater than the Y-displacement of the plurality of successive curves in the proximal portion.
